(19) United States
(12) Patent Application Publication
(10) Pub. No.: US 2023/0384445 A1
(43) Pub. Date: Nov. 30, 2023

(54) ULTRASONIC IMAGING SYSTEM AND METHOD

(71) Applicant: SHENZHEN MINDRAY BIO-MEDICAL ELECTRONICS CO., LTD., Shenzhen (CN)

(72) Inventors: Chongchong GUO, SHENZHEN (CN); Jing LIU, SHENZHEN (CN); Bo YANG, SHENZHEN (CN); Lei LI, SHENZHEN (CN)

(21) Appl. No.: 18/203,512

(22) Filed: May 30, 2023

(30) Foreign Application Priority Data
May 31, 2022 (CN) 202210612151.7

(51) Int. Cl. G01S 15/89; G01S 7/52

(52) U.S. Cl. CPC G01S 15/8995; G01S 15/8925; G01S 7/52095

(57) ABSTRACT

Disclosed are an ultrasonic imaging system and method thereof. An ultrasonic probe is controlled to transmit ultrasonic waves to a region of interest at a plurality of steering angles respectively. Ultrasonic echoes are received to obtain the channel echo data. The channel echo data corresponding to at least part of the steering angles are beam-formed with the same receiving-line grid to obtain the beam-formed data.

### CROSS-REFERENCE TO RELATED APPLICATIONS

This application is based on and claims priority to and benefits of Chinese Patent Application No. 202210612151.7, filed on May 31, 2022. The entire content of the above-referenced application is incorporated herein by reference.

### TECHNICAL FIELD

The present disclosure relates to ultrasonic imaging, in particular to ultrasonic imaging systems and methods.

### BACKGROUND

Spatial compounding imaging technology is the most widely used in composite ultrasonic imaging technology which is important in the field of ultrasonic imaging. Because of suppression of image noise, spatial compounding imaging technology plays an important role in improving the overall quality of images; therefore, most ultrasonic imaging systems currently used is configured with and default to using such technology in ultrasonic imaging.

There is still room for improvement in the current composite ultrasonic imaging technology.

### SUMMARY

In one embodiment, an ultrasonic imaging system is provided, which may include:
an ultrasonic probe having a plurality of array elements;
a transmitting and receiving control circuit configured to control the ultrasonic probe to transmit ultrasonic waves to a region of interest and receive ultrasonic echoes of the transmitted ultrasonic waves, to obtain channel echo data;
a processor configured to generate an ultrasonic image according to the channel echo data; and
a display configured to display the ultrasonic image;
where, the transmitting and receiving control circuit is configured to:
control the ultrasonic probe to transmit the ultrasonic waves at a plurality of steering angles to the region of interest, respectively; and
control the ultrasonic probe to receive the ultrasonic echoes of the ultrasonic waves transmitted at the plurality of steering angles, to obtain multiple groups of channel echo data, where each group of channel echo data is obtained by the ultrasonic echoes of the ultrasonic waves transmitted at one of the plurality of steering angles; and
where, the processor is configured to:
beam-form the multiple groups of channel echo data with a receiving-line grid including a plurality of grid points to obtain multiple groups of beam-formed data, where at least two of the multiple groups of channel echo data are beam-formed with a same receiving-line grid; and
generate the ultrasonic image based on the multiple groups of beam-formed data.

In one embodiment, for one grid point of the receiving-line grid for one steering angle, the processor is configured to:
determines a receiving aperture based on a physical spatial position of the one grid point and the one steering angle; and
beam-form, based on the determined receiving aperture, the group of channel echo data obtained by the ultrasonic echoes of the ultrasonic waves transmitted at the one steering angle.

In one embodiment, the processor determining a receiving aperture based on a physical spatial position of the one grid point and the one steering angle may include:
determining an intersection point of a straight line passing through the physical spatial position of the one grid point and forming an angle equal to the one steering angle with respect to a normal of the ultrasonic probe; and
determining the receiving aperture based on the intersection point.

In one embodiment, the processor is further configured to perform a spatial compounding on the groups of beam-formed data obtained by the beam-forming with the same receiving-line grid.

In one embodiment, the same receiving-line grid is the one corresponding to the steering angles being 0.

In one embodiment, the processor generating the ultrasonic image based on the multiple groups of beam-formed data may include performing digital scan conversion on the beam-formed data to obtain ultrasonic image pixel data for display, and the display may display the ultrasonic image pixel data.

In one embodiment, an ultrasonic imaging method is provided, which may include:
transmitting, via an ultrasonic probe, ultrasonic waves at a plurality of steering angles to a region of interest, respectively;
receiving, via the ultrasonic probe, ultrasonic echoes of the ultrasonic waves transmitted at the plurality of steering angles, to obtain multiple groups of channel echo data, where each group of channel echo data is obtained by the ultrasonic echoes of the ultrasonic waves transmitted at one of the plurality of steering angles;
beam-forming the multiple groups of channel echo data with a receiving-line grid including a plurality of grid points to obtain multiple groups of beam-formed data, where at least two of the multiple groups of channel echo data are beam-formed with a same receiving-line grid; and
generating an ultrasonic image based on the multiple groups of beam-formed data.

In one embodiment, beam-forming the multiple groups of channel echo data with receiving-line grid including a plurality of grid points may include:
for one grid point of the receiving-line grid for one steering angle:
   determining a receiving aperture based on a physical spatial position of the one grid point and the one steering angle; and
beam-forming, based on the determined receiving aperture, the group of channel echo data obtained by the ultrasonic echoes of the ultrasonic waves transmitted at the one steering angle.

In one embodiment, determining a receiving aperture based on a physical spatial position of the one grid point and the one steering angle may include:
determining an intersection point of a straight line passing through the physical spatial position of the one grid point and forming an angle equal to the one steering angle with respect to a normal of the ultrasonic probe; and
determining the receiving aperture based on the intersection point.

In one embodiment, the same receiving-line grid may be determined according to scan mode.

In one embodiment, the same receiving-line grid may be a receiving-line grid under a Cartesian coordinate system when the scan mode is linear array scan mode; or, the same receiving-line grid may be a receiving-line grid under a polar coordinate system when the scan mode is convex array scan mode or sector scan mode.

In one embodiment, the same receiving-line grid may be the one corresponding to the steering angles being 0.

In one embodiment, the method may further include performing a spatial compounding on the groups of beam-formed data obtained by the beam-forming with the same receiving-line grid.

In one embodiment, performing a spatial compounding on the groups of beam-formed data obtained by the beam-forming with the same receiving-line grid may include:
for one grid point of the same receiving-line grid, performing spatial compounding on the groups of beam-formed data by averaging, weighted averaging, taking a maximum value or taking a median value.

In one embodiment, generating an ultrasonic image based on the multiple groups of beam-formed data may include:
performing digital scan conversion on the beam-formed data to obtain ultrasonic image pixel data for display.

In one embodiment, a computer-readable storage medium is provided, which may include a program capable of being executed by a processor to:
beam-form multiple groups of channel echo data with receiving-line grid including a plurality of grid points to obtain multiple groups of beam-formed data, the multiple groups of channel echo data being obtained by transmitting ultrasonic waves at a plurality of steering angles and receiving ultrasonic echoes of the ultrasonic waves transmitted at the plurality of steering angles, and each group of channel echo data being obtained by the ultrasonic echoes of the ultrasonic waves transmitted at one of the plurality of steering angles, where at least two of the multiple groups of channel echo data are beam-formed with a same receiving-line grid; and
generate an ultrasonic image based on the multiple groups of beam-formed data.

According to the ultrasonic imaging system, method and computer readable storage medium disclosed in the above embodiments, the ultrasonic probe may be controlled to transmit the ultrasonic waves at a plurality steering angles to the region of interest and receive the ultrasonic echoes to obtain the channel echo data, and then the channel echo data corresponding to at least part of the steering angles may be beam-formed with the same receiving-line grid to obtain the beam-formed data; therefore, it is not necessary to transform the receiving-line grid corresponding to different steering angles to the same coordinate during spatial compounding. Accordingly, at least a part of the issues due to the transformation can be avoided.

### BRIEF DESCRIPTION OF THE DRAWINGS

FIG. 1 is a schematically structural diagram of an ultrasonic imaging system according to an embodiment;

FIG. 2(a) is a schematic diagram of beam-formed points of a linear array probe according to an embodiment;

FIG. 2(b) is a schematic diagram of beam-formed points of a convex array probe according to an embodiment;

FIG. 3(a) is a schematic diagram showing linear array scanning from three steering angles according to an embodiment;

FIG. 3(b) is a schematic diagram showing convex array scanning from three steering angles according to an embodiment;

FIG. 4 is a schematic diagram of spatial compounding according to an embodiment;

FIG. 5 is a schematic diagram of performing compounding based on pixel points according to an embodiment;

FIG. 6 is a schematic diagram of the same receiving-line grid corresponding to different steering angles according to an embodiment;

FIG. 7(a) is a schematic diagram of determining the receiving aperture according to an embodiment;

FIG. 7(b) is a schematic diagram of determining the receiving aperture according to another embodiment;

FIG. 8(a) is a schematic diagram showing, in the case of under a first steering angle shown in FIG. 3(a), intersection points corresponding to some receiving points (grid points) of a receiving-line grid which is the same as the one corresponding to a second steering angle, the intersection points being used for determining the receiving aperture;

FIG. 8(b) is a schematic diagram showing, in the case of under a first steering angle shown in FIG. 3(b), intersection points corresponding to some receiving points (grid points) of a receiving-line grid which is the same as the one corresponding to a second steering angle, the intersection points being used for determining the receiving aperture;

FIG. 9(a), FIG. 9(b) and FIG. 9(c) are schematic diagrams of the same receiving-line grid at each steering angle under the linear array scan mode, the sector scan mode and the convex array scan mode, respectively;

FIG. 10 is a flowchart of an ultrasonic imaging method according to another embodiment; and

FIG. 11 is a flowchart of controlling the ultrasonic probe to receive the ultrasonic echoes in the same receiving-line grid at each of the steering angles to convert the echoes into the channel echo data according to an embodiment.

### DETAILED DESCRIPTION

The present disclosure will be further described in detail below through specific embodiments with reference to the accompanying drawings. Common or similar elements are referenced with like or identical reference numerals in different embodiments. Many details described in the following embodiments are for better understanding the present disclosure. However, those skilled in the art can realize with minimal effort that some of these features can be omitted in different cases or be replaced by other elements, materials and methods. For clarity some operations related to the present disclosure are not shown or illustrated herein so as to prevent the core from being overwhelmed by excessive descriptions. For those skilled in the art, such operations are not necessary to be explained in detail, and they can fully understand the related operations according to the description in the specification and the general technical knowledge in the art.

In addition, the features, operations or characteristics described in the specification may be combined in any suitable manner to form various embodiments. At the same time, the steps or actions in the described method can also be sequentially changed or adjusted in a manner that can be apparent to those skilled in the art. Therefore, the various sequences in the specification and the drawings are only for the purpose of describing a particular embodiment, and are not to be an order of necessity, unless otherwise stated one of the sequences must be followed.

The serial numbers of components herein, such as “first”, “second”, etc., are only used to distinguish the described objects and do not have any order or technical meaning. The terms “connected”, “coupled” and the like here include direct and indirect connections (coupling) unless otherwise specified.

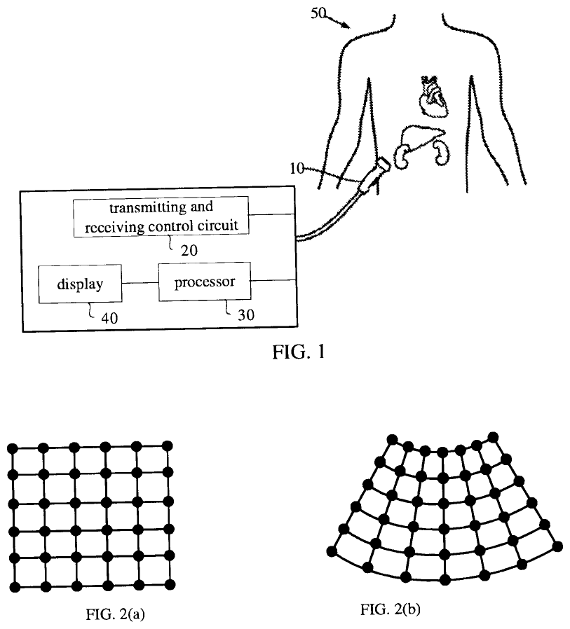

Please refer to FIG. 1, an ultrasonic imaging system in an embodiment may include an ultrasonic probe 10, a transmitting and receiving control circuit 20 and a processor 30. The ultrasonic imaging system in another embodiment may also include a display 40. The following describes the components of the system.

The ultrasonic probe 10 may be used to transmit ultrasonic waves to a region of interest and receive corresponding ultrasonic echoes to convert the echoes into channel echo data. In some embodiments, the ultrasonic probe 10 may include a plurality of array elements to realize mutual conversion of electrical pulse signals and the ultrasonic waves, implementing transmissions of the ultrasonic waves to a biological tissue under examination 50 (biological tissue in human body or animal body) and reception of ultrasonic echoes returned from the tissue to obtain ultrasonic echo signals to convert them into the channel echo data of the ultrasonic waves. The plurality of array elements included in the ultrasonic probe 10 may be arranged into a row to form a linear array, or into a two-dimensional matrix to form a planar array. The plurality of array elements may also form a convex array. The array elements may either transmit the ultrasonic waves according to excitation electrical signals, or convert the received ultrasonic echoes into electrical signals. Thus each of the array elements may be used to transmit the ultrasonic waves to the biological tissue in the region of interest, and be used to receive the ultrasonic echoes from the tissue. In ultrasonic detection, a transmitting sequence and a receiving sequence may be used to control which array elements for transmitting the ultrasonic waves (referred to as transmitting array elements) and which array elements for receiving the ultrasonic echoes (referred to as receiving array elements), or control the plurality of array elements in time slots for transmitting the ultrasonic waves or receiving the ultrasonic echoes. All the array elements participating in the transmission of the ultrasonic waves may be excited by the electrical signals simultaneously, thus transmitting the ultrasonic waves; alternatively, they may be excited by several electrical signals with a certain time interval to continuously transmit the ultrasonic waves with a certain time interval. Regarding a minimum processing region of the biological tissue under examination 50 (in which the ultrasonic waves are transmitted and received) as a position point within the tissue, the ultrasonic waves will produce different reflection when they reach each position point of the biological tissue under examination 50 due to different tissue acoustic impedance at different position points; and the reflected ultrasonic waves may be picked up by the receiving array elements. Each of the receiving array elements may receive ultrasonic echoes from multiple position points. The ultrasonic echoes of different position points received by each of the receiving array elements may form different channel echo data. The multiple channel echo data outputted by each of the receiving array elements may form a group of channel echo data corresponding to the receiving array element. For a receiving array element, the distance from it to different position points of the biological tissue under examination 50 may be different, so the time for the ultrasonic echoes reflected from each of the position point to the array element may also be different; accordingly, a correspondence between the ultrasonic echoes and the position points may be determined according to the time it takes for the ultrasonic echoes to arrive at the array elements.

It should be noted that the “channel echo data” involved herein is the data before beam synthesis processing corresponding to a channel of the ultrasonic imaging system (the channel corresponds to one or more array elements).

In some examples, the region of interest may be selected by a user. For example, when a general ultrasonic image is displayed on the display 40, the user may select the region of interest on the general ultrasonic image. In some examples, the location of the region of interest may also be automatically determined on a base ultrasonic image by the processor 30 based on a relevant machine recognition algorithm. In some examples, the region of interest may be obtained by semi-automatic detection. For example, the location of the region of interest on the base ultrasonic image based on the machine recognition algorithm may first be automatically detected by the processor 30, and then be modified or corrected by the user to obtain a more accurate location of the region of interest.

The transmitting and receiving control circuit 20 may be used to control the ultrasonic probe 10 to transmit the ultrasonic waves and receive corresponding ultrasonic echoes to convert them into channel echo data. In some embodiments, the transmitting control circuit in the transmitting and receiving control circuit 20 may be used to control the ultrasonic probe 10 to transmit the ultrasonic waves to the biological tissue 50 (such as the region of interest), and the receiving control circuit of the transmitting and receiving control circuit 20 may be used to control the ultrasonic probe 10 to receive the ultrasonic echoes reflected by the tissue to which the ultrasonic waves are transmitted to convert them into the channel echo data corresponding to the ultrasonic waves. In some embodiments, the transmitting and receiving control circuit 20 may be used to generate the transmitting sequence and the receiving sequence, and output them to the ultrasonic probe 10. The transmitting sequence may be used to control part or all of the plurality of array elements of the ultrasonic probe 10 to transmit the ultrasonic waves to the biological tissue 50. Parameters of the transmitting sequence may include the number of the transmitting array elements and transmitting parameters of the ultrasonic waves (such as amplitude, frequency, number of transmitting waves, transmitting interval, transmitting steering angle, waveform and/or focusing position, etc.). The receiving sequence may be used to control part or all of the plurality of array elements to receive echoes reflected by the tissue to which the ultrasonic waves are transmitted. Parameters of the receiving sequence may include the number of the receiving array elements and receiving parameters of the echoes (such as receiving aperture and depth, etc.). With different applications of the ultrasonic echoes or different images generated by the ultrasonic echoes, the parameters of the ultrasonic waves of the transmitting sequence and the echo parameters of the receiving sequence may be various.

The processor 30 may be used to generate ultrasonic images based on the channel echo data. For example, after obtaining the channel echo data, the processor 30 may acquire required parameters or images obtained by correlation algorithms. To be specific, after obtaining the channel echo data, the processor 30 may obtain beam-formed data by beam forming, and perform spatial compounding on the beam-formed data to obtain beam-formed data. In some embodiments, the processor 30 may be a device that is used to interpret computer instructions and process data in computer software, including but being not limited to a central processing unit (CPU), a micro controller unit (MCU), a field-programmable gate array (FPGA) and a digital signal processing (DSP) device. In some embodiments, the processor 30 may be used to execute each computer application in a non-temporary computer-readable storage medium to perform the corresponding ultrasonic imaging process.

The display 40 may be used to display information, such as parameters and images calculated by the processor 30. A person skilled in the art should understand that in some embodiments, instead of integrating with the display 40, the ultrasonic imaging system per se may connect to a computer device (e.g. a computer) to display the information by a display module of the computer device (e.g. a display screen).

The above are some instructions for the ultrasonic imaging system. Here is an example to describe how to process the channel echo data of the ultrasonic waves to obtain the ultrasonic image.

The ultrasonic probe 10 may receive signals reflected from the region of interest to which the ultrasonic waves have been transmitted, namely the ultrasonic echo signals, and then convert the ultrasonic echo signals (or, the ultrasonic echoes) into the channel echo data of the ultrasonic waves. Beam-forming may refer to reconstructing and converting the channel echo data from channel domain (for example, the data dimension thereof is: time direction*the number of channels*the number of transmissions) into beam domain (i.e. the beam-formed data, for example, the data dimension thereof is: the number of longitudinal points*the number of horizontal points, the points being the one in actual physical space); and beam-formed points may refer to each output point in the beam domain. For example, FIG. 2(a) schematically shows the beam-formed points of a linear array probe, and FIG. 2(b) schematically shows the beam-formed points of a convex array probe. The beam-forming may be performed at pre-determined points, thereby obtaining the beam-formed data at said pre-determined points. The points on which the beam-forming will be performed may form a grid, as shown in FIG. 2(a) and FIG. 2(b). The grid may be referred to as the receiving-line grid. Accordingly, beam forming may refer to reconstructing and converting the channel echo data from the channel domain into the beam domain. Specifically, the data dimension of the channel echo data of the ultrasonic waves may be Nₜ*N_{ch}*Nₜₓ, where N t may represent the time direction of the propagation of the ultrasonic waves, N_{ch} may represent the number of channels, and Nₜₓ may represent the number of transmissions; and the data dimension may turn into the data of the beam domain after beam forming, and the data dimension of the data of the beam domain may be N_{rang}*Nᵤₛₗ, where N_{rang} may represent a vertical depth point of the receiving-line grid, and may represent the number of horizontal points of the receiving-line grid.

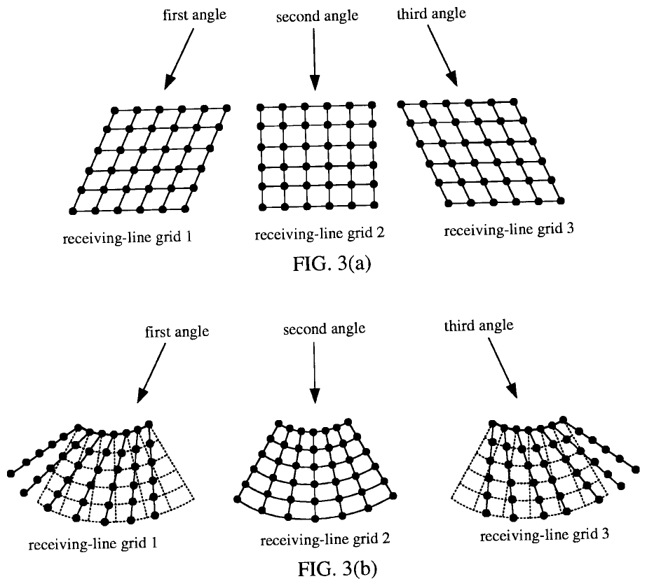

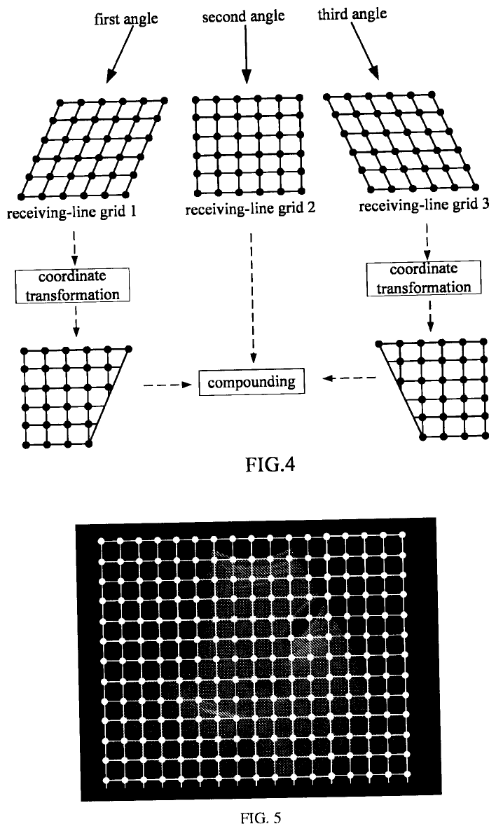

When performing multi-steering angle scanning or transmitting the ultrasonic waves at multiple steering angles, for example the number of the steering angles is N_{rangle}, N_{rangle} receiving-line grids may be generated correspondingly. FIG. 3(a) is an example of linear array scanning with three steering angles, forming three receiving-line grids corresponding to the three steering angles respectively, where a first steering angle corresponds to a first receiving-line grid, a second steering angle corresponds to a second receiving-line grid and a third corresponds to a third receiving-line grid; and FIG. 3(b) is an example of convex array scanning with three steering angles, forming three receiving-line grids corresponding to the three steering angles respectively, where a first steering angle corresponds to a first receiving-line grid, a second steering angle corresponds to a second receiving-line grid and a third corresponds to a third receiving-line grid. When there are receiving-line grids corresponding to the multiple steering angles, a spatial compounding technique may be employed on the data points of the receiving-line grid. Taking the example shown in FIG. 3(a), FIG. 4 is an example of spatial compounding. Specifically, when transmitting in a deflected manner, receiving lines are along the steering angle direction of transmitting lines; in this way, the formed receiving-line grid is also corresponding to the steering angle, and the receiving-line grids of different steering angles are different. In order to perform compounding, it is necessary to convert the coordinates of the receiving-line grids of different steering angles so that the grid points (i.e. beam-formed points) of the converted receiving-line grids can correspond to the different steering angles so as to compound. Finally, by means of digital scan conversion, the coordinate system of the receiving-line grids is transformed to the coordinate system of the pixel points; that is, the beam-formed data is transformed to the coordinate system of the pixels on screen by operations including coordinate transformation to obtain ultrasonic image pixel data in pixel coordinate system, and then output to the display for display.

Data has been processed with several times of data reconstruction and transformation, including from the channel echo data to line data or point data of the receiving-line grid, then to the beam-formed data, and finally to the ultrasonic image pixel data used for display. Coordinate transformation is needed for every transformation, and other processing including interpolation may be involved. These processing, especially spatial compounding, has a smoothing effect on the data in essence, leading to side effects on the ultrasonic image such as making the image blurred. As illustrated in FIG. 4 above, the receiving-line grids corresponding to the three steering angles is in different coordinate systems. In the figure, coordinate transformation is performed on the receiving-line data of the first and third steering angles, during which interpolation is carried out to form new grid points so as to align with the receiving-line grid points at the second steering angle, and then the three groups of data therefrom are composite. Spatial compounding can reduce image noise, however it may bring the side effect of blurred image.

One solution is making the grid points of data outputted by beam-forming be the pixel points. This way, the spatial compounding is based on the pixel points accordingly without coordinate transformation. This idea can solve the above problem well. However, as shown in FIG. 5, the solution is based on the pixel points under the displayed Cartesian coordinate system, so there will be some problems: (1) a very high requirement on each image processing algorithm of the processor so as to adapt to arbitrary number of points at each depth position, because the algorithm is influenced greatly by the different number of pixel points at each depth position within the range of effective image region (the effective imaging region shown in the figure); (2) extremely complex and high cost of the ultrasonic imaging apparatus due to requirements of extremely high flexibility and data processing ability for the apparatus, where such requirements are because the whole imaging system is based on pixel points, and the whole signal and image processing flow is based on the “pixel points” without concept of receiving lines; (3) decrease in the transverse sampling rate of near-field image under many scan modes (such as convex array scanning, phased array scanning, extended scanning, etc.) by using the coordinate system of the pixel points, compared with using the polar coordinate system which is more suitable and friendly to process data, and even worse quality of the near-field image due to under-sampling and wasting computational power in far-field image due to too high sampling rate in severe conditions.

In the embodiments of present disclosure, ultrasonic imaging systems and methods including improvement of composite imaging are proposed. When the ultrasonic waves are transmitted at multiple steering angles, the receiving-line grid for at least part of the steering angles is the same receiving-line grid during the beam-forming, in other words, the beam-forming for the channel echo data of the ultrasonic waves transmitted in at least part of the different steering angles is carried out with the same receiving-line grid. This way, there is no need to transform the receiving-line grid corresponding to the different steering angles to the same coordinate in spatial compounding, and no loss of data due to interpolation is needed during compounding, which will be described in detail below.

In some embodiments, the transmitting and receiving control circuit 20 may control the ultrasonic probe 10 to transmit the ultrasonic waves at multiple steering angles to the region of interest, respectively; and the transmitting and receiving control circuit 20 may control the ultrasonic probe 10 to receive the ultrasonic echoes of the ultrasonic waves transmitted at the plurality of steering angles, thereby obtaining multiple groups of channel echo data, where each group of channel echo data may be obtained by the ultrasonic echoes of the ultrasonic waves transmitted at one of the plurality of steering angles. It should be noted that the steering angle involved herein may refer to the transmitting steering angle of the ultrasonic waves; for example, the transmitting steering angle of the ultrasonic waves may be defined as an angle between the transmitting direction of the ultrasonic waves and the normal direction of the ultrasonic probe.

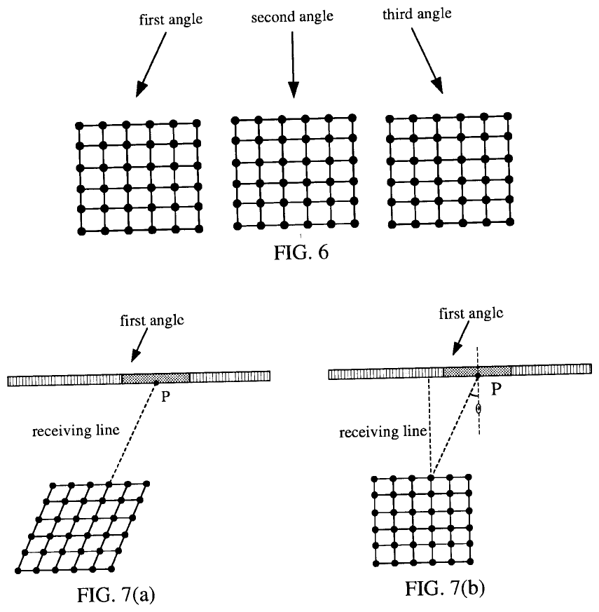

As described above, different steering angles may correspond to different receiving-line grids, such as the example shown in FIG. 3. In some embodiments of the present disclosure, the receiving-line grids corresponding to at least part (e.g., at least two) of the steering angles are identical, as the example shown in FIG. 6. The receiving-line grids corresponding to the first steering angle, the second steering angle and the third steering angle are the same. In these embodiments, the processor may beam-form the multiple groups of channel echo data with receiving-line grid including a plurality of grid points to obtain multiple groups of beam-formed data, where at least two of the multiple groups of channel echo data are beam-formed with a same receiving-line grid.

Referring to FIG. 7(a), for each receiving point (i.e. the position point, the beam-formed point or the grid point), the receiving aperture (represented by the shade array element in the figure) is determined by the intersection point (e.g. P in the figure) of the receiving line on which the receiving point is located and the ultrasonic probe, such that an output after being beam-formed can be obtained; in this way, different steering angles finally correspond to different receiving-line grids. In some embodiments of the present disclosure, a receiving-line grid may be predetermined, and the channel echo data corresponding to at least two of the steering angles may be beam-formed based on the same predetermined receiving-line grid. Referring to FIG. 7(b), it is an example of making the receiving-line grid corresponding to each of the steering angles to be the receiving-line grid shown in FIG. 6, that is: in order to make the same receiving point to obtain identical beam-formed output, an intersection point of the ultrasonic probe (e.g. P′ in the figure) is determined by the receiving point and the corresponding steering angle in some embodiments, and this intersection point can make an steering angle between a straight line determined by the intersection point and the receiving point and the normal of the ultrasonic probe at the intersection point to be equal to the aforesaid corresponding steering angle, that is, the steering angle θ shown in the figure equals to the first steering angle. In other words, an intersection point of a straight line passing through the physical spatial position of the grid point (receiving point) and forming an angle equal to the steering angle with respect to a normal of the ultrasonic probe may be determined.

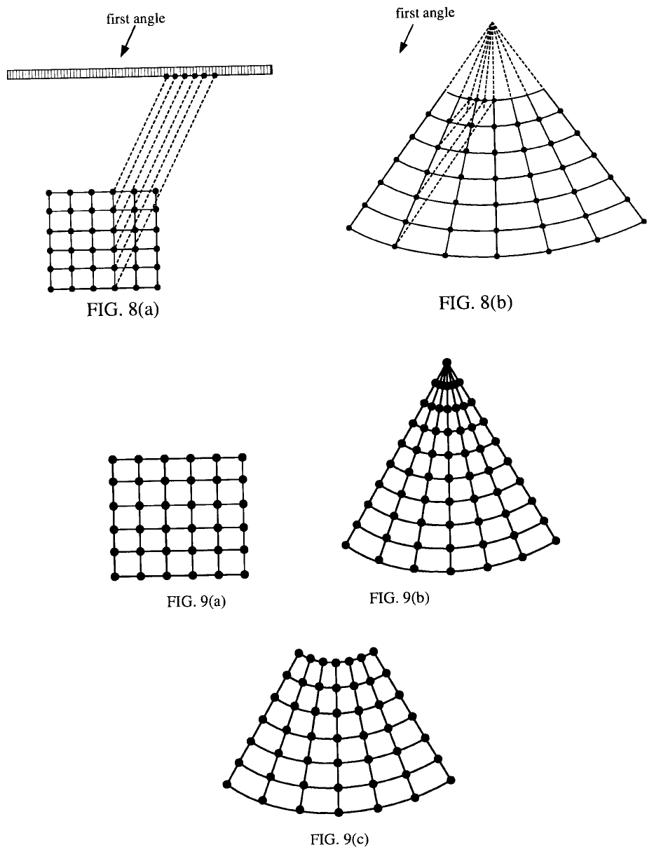

After searching out such intersection point, the receiving aperture may then be determined. FIG. 8(a) is an example in which in the case of under a first steering angle shown in FIG. 3(a), intersection points corresponding to some receiving points (grid points) of a receiving-line grid which is the same as the one corresponding to a second steering angle, the intersection points being used for determining the receiving aperture. FIG. 8 is an example of a linear scan; and sector scanning and convex array scanning are similar. Taking convex array scanning as an example, FIG. 8(b) is an example in which in the case of under a first steering angle shown in FIG. 3(b), intersection points corresponding to some receiving points (grid points) of a receiving-line grid which is the same as the one corresponding to a second steering angle, the intersection points being used for determining the receiving aperture.

Therefore, in some embodiments, for any one grid point (taking a grid point a as an example) of the same receiving-line grid for any one steering angle (taking an steering angle A as an example), the processor may determine the receiving aperture of the grid point a for the steering angle A based on the physical spatial position of the grid point a and the steering angle A. In some embodiments, the processor 30 may determine an intersection point of a straight line passing through the physical spatial position of the one grid point a and forming an angle equal to the one steering angle A with respect to a normal of the ultrasonic probe. The processor 30 may determine the receiving aperture of the grid point a (referred to as the receiving aperture Ra) based on the intersection point; for example, by taking the above intersection point as the center of the receiving aperture Ra and determining the diameter of the receiving aperture Ra based on a predetermined diameter parameter, the receiving aperture Ra of the grid point a may thus be determined. The processor 30 may beam-form, based on the determined receiving aperture Ra, the group of channel echo data obtained by the ultrasonic echoes of the ultrasonic waves transmitted at the one steering angle, thereby obtaining the beam-formed data corresponding to the grid point a at the steering angle A.

The obtained beam-formed data at the receiving-line grid may be further processed to generate at least a part of a frame of ultrasonic image of the region of interest. It should be noted that, as described above, the points (grid points) in the receiving-line grid are the points at which the beam-forming will be performed to obtain the beam-formed data thereat, but not pixel points of ultrasound image to be displayed on the display.

In some embodiments, the processor 30 may further perform a spatial compounding on the groups of beam-formed data obtained by the beam-forming with the same receiving-line grid.

In some embodiments, when the processor 30 performs spatial compounding on the beam-formed data in the same receiving-line grid at the different steering angles, it is not necessary to transform coordinates according to the beam-formed data. Since the receiving-line grid corresponds to the different steering angles is identical, it is unnecessary to perform coordinate transformation of the grid points as shown in FIG. 4 above when performing spatial compounding.

In some embodiments, when the processor 30 performs spatial compounding on the beam-formed data in the same receiving-line grid at the different steering angles, there is no data loss. Since the receiving-line grid corresponds to the different steering angles is identical, there is no need to interpolate a new grid point without loss of data in the process of spatial compounding.

In some embodiments, for any one grid point of the same receiving-line grid, the processor 30 may perform spatial compounding on the beam-formed data at each of the steering angles by means of averaging, weighted averaging, taking a maximum value, or taking a median value.

The above are some description for performing beam forming and spatial compounding on the channel echo data.

For the “same receiving-line grid” involved as mentioned above, in some embodiments, the same receiving-line grid corresponding to the different steering angles under different scan modes may be different. For example, the same receiving-line grid may be the one under a Cartesian coordinate system when the scan mode (or scanning mode) is linear array scan mode. FIG. 9(a) is an example of the same receiving-line grid corresponding to the different steering angles under the linear array scan mode. For another example, the receiving-line grid may be the one under a polar coordinate system when the scan mode is convex array scan mode or sector scan mode. FIG. 9(b) is an example of the same receiving-line grid corresponding to the different steering angles under the sector scan mode, and FIG. 9(c) is an example of the same receiving-line grid corresponding to the different steering angles under the convex array scan mode.

Therefore, in some embodiments, the processor 30 may obtain the scan mode and may obtain the same receiving-line grid corresponding to the different steering angles according to the obtained scan mode. For example, when the scan mode is the linear array scan mode, the same receiving-line grid is a receiving-line grid under the Cartesian coordinate system; and when the scan mode is the convex array scan mode or the sector scan mode, the same receiving-line grid is a receiving-line grid under the polar coordinate system.

In some embodiments, the same receiving-line grid is the one corresponding to the steering angle being 0.

In some embodiments, after obtaining the beam-formed data, the processor 30 may generate an ultrasonic image based on the beam-formed data, and the display 40 may display the ultrasonic image. In some embodiments, the processor 30 may perform digital scan conversion (such as coordinate transformation) on the beam-formed data to obtain ultrasonic image pixel data for display, and the display 40 may display the corresponding ultrasonic image based on the ultrasonic image pixel data.

The above is some description of the ultrasonic imaging system.

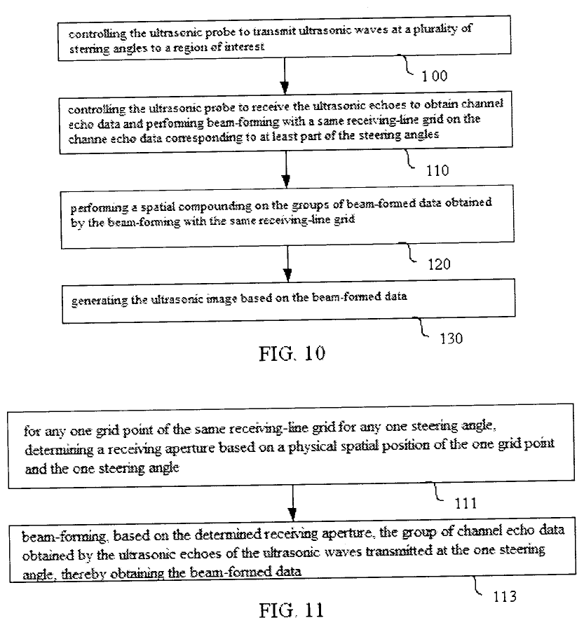

An ultrasonic imaging method may also be provided herein. Referring to FIG. 10, the ultrasonic imaging method of some embodiments may include the following steps:

Step 100: controlling the ultrasonic probe to transmit ultrasonic waves at a plurality of steering angles to the region of interest.

Step 110: controlling the ultrasonic probe to receive the ultrasonic echoes of the ultrasonic waves transmitted at the plurality of steering angles, thereby obtaining multiple groups of channel echo data, where each group of channel echo data is obtained by the ultrasonic echoes of the ultrasonic waves transmitted at one of the plurality of steering angles, and beam-forming the multiple groups of channel echo data with receiving-line grid comprising a plurality of grid points to obtain multiple groups of beam-formed data, where at least two of the multiple groups of channel echo data are beam-formed with a same receiving-line grid.

Referring to FIG. 11, the step 110 may include:

Step 111: for any one grid point of the same receiving-line grid for any one steering angle, determining a receiving aperture based on a physical spatial position of the one grid point and the one steering angle. In some embodiments, by the physical spatial position of said any one grid point and said any one steering angle, an intersection point of a straight line passing through the physical spatial position of the one grid point and forming an angle equal to the one steering angle with respect to a normal of the ultrasonic probe may be determined. In step 111, the receiving aperture may be determined based on the intersection point.

Step 113: beam-forming, based on the determined receiving aperture, the group of channel echo data obtained by the ultrasonic echoes of the ultrasonic waves transmitted at the one steering angle, thereby obtaining the beam-formed data.

For example, in some embodiments, for any one grid point (taking a grid point a as an example) of the same receiving-line grid at any one steering angle (taking an steering angle A as an example), the receiving aperture of the grid point a at the steering angle A may be calculated based on the physical spatial position of the grid point a and the steering angle A. In some embodiments, the intersection point of the grid point a and the array element of the ultrasonic probe 10 may be determined based on the physical spatial position of the grid point a and the steering angle A, where the intersection point makes an steering angle between a straight line determined by the intersection point and the grid point a and the normal of the ultrasonic probe 10 at the intersection point be the steering angle A. The receiving aperture of the grid point a (referred to as the receiving aperture Ra) may be determined based on the intersection point. The ultrasonic probe may then be controlled to receive the ultrasonic echoes with the receiving aperture Ra to convert the echoes into the channel echo data, thereby obtaining the channel echo data corresponding to the grid point a at the steering angle A.

Step 120: performing a spatial compounding on the groups of beam-formed data obtained by the beam-forming with the same receiving-line grid.

In some embodiments, when performing spatial compounding on the beam-formed data in the same receiving-line grid at the different steering angles in step 120, it is unnecessary to transform coordinates according to the beam-formed data. Since the receiving-line grid corresponds to the different steering angles is identical, it is unnecessary to perform coordinate transformation of the grid points as shown in FIG. 4 above when performing spatial compounding.

In some embodiments, when performing spatial compounding on the beam-formed data in the same receiving-line grid at the different steering angles in step 120, there is no data loss. Since the receiving-line grid corresponds to the different steering angles is identical, there is no need to interpolate a new grid point without loss of data in the process of spatial compounding.

In some embodiments, for any one grid point of the same receiving-line grid, spatial compounding on the beam-formed data at each of the steering angles may be performed by means of averaging, weighted averaging, taking a maximum value, or taking a median value in step 120.

Step 130: generating the ultrasonic image based on the beam-formed data.

For the “same receiving-line grid” involved in the ultrasonic imaging method, in some embodiments, the same receiving-line grid corresponding to the different steering angles under different scan modes may be different. Accordingly, the ultrasonic imaging method in some embodiments may further include: obtaining a scan mode, and obtaining the same receiving-line grid corresponding to each of the steering angles according to the obtained scan mode. For example, when the scan mode is the linear array scan mode, the same receiving-line grid is the one under the Cartesian coordinate system; and when the scan mode is the convex array scan mode or the sector scan mode, the same receiving-line grid is the one under the polar coordinate system.

In some embodiments, the same receiving-line grid is the one corresponding to the steering angle being 0.

The above is some description of the ultrasonic imaging method.

In some embodiments of the present disclosure, the grid points of the receiving-line grid remain unchanged, that is, when scanning at the multiple steering angles, the grid points remain unchanged, beam forming is carried out on the same grid and then compounding and subsequent signal processing are performed.

With the ultrasonic imaging system and method according to the embodiments of the present disclosure, the receiving-line grid at multiple steering angles is identical, so that it is unnecessary to perform coordinate transformation in spatial compounding, avoiding image blurring caused by traditional spatial compounding.

In addition, the receiving-line grid at different steering angles in traditional spatial compounding results in “missing corner” after coordinate transformation; for example, there is a corner losing in the receiving-line grid at the first steering angle in FIG. 4 after coordinate transformation, and a corner losing in the receiving-line grid at the second steering angle after coordinate transformation. Actually, in the receiving-line grid, there are data compounding of three steering angles in the middle area, and data compounding of only two steering angles occurs in the missing corner. Hence, in spatial compounding, there needs special considerations and optimization processing for the calculation of a compounding coefficient, including the transition of images and the setting of transition zones, which also affect resulted images, resulting in degradation of image performance. However, according to some embodiments of the present disclosure, each receiving point is performed with data compounding at all steering angles without data loss, so there is no need to study advanced algorithms for compounding.

The present disclosure is illustrated with reference to various exemplary embodiments. However, those skilled in the art may recognize that the exemplary embodiments can be changed and modified without departing from the scope of the present disclosure. For example, various operation steps and components used to execute the operation steps may be implemented in different ways (for example, one or more steps may be deleted, modified, or combined into other steps) according to specific application(s) or any number of cost functions associated with the operation of the system.

In the above embodiments, it may be done in whole or in part by software, hardware, firmware, or any combination thereof. In addition, as understood by those skilled in the art, the principles herein may be reflected in a computer program product on a computer-readable storage medium that is preloaded with computer-readable program code. Any tangible, non-temporary computer-readable storage medium can be used, including magnetic storage devices (hard disks, floppy disks, etc.), optical storage devices (CD-ROMs, DVDs, Blu Ray disks, etc.), flash memory and/or the like. The computer program instructions may be loaded onto a general purpose computer, a special purpose computer, or other programmable data processing device to form a machine, so that these instructions executed on a computer or other programmable data processing device can form a device that realizes a specified function. These computer program instructions may also be stored in a computer-readable memory that can instruct a computer or other programmable data processing device to run in a specific way, so that the instructions stored in the computer-readable memory can form a manufacturing product, including a realization device to achieve a specified function. The computer program instructions may also be loaded onto a computer or other programmable data processing device to execute a series of operating steps on the computer or other programmable device to produce a computer-implemented process, so that instructions executed on the computer or other programmable device can provide steps for implementing a specified function.

Although the principles herein have been shown in various embodiments, many modifications to structures, arrangements, proportions, elements, materials, and components that are specifically adapted to specific environmental and operational requirements may be used without deviating from the principles and scope of the present disclosure. These and other modifications and amendments will be included in the scope of the present disclosure.

The foregoing specific description has been illustrated with reference to various embodiments. However, those skilled in the art will recognize that various modifications and changes can be made without departing from the scope of the present disclosure. Accordingly, the present disclosure is illustrative rather than restrictive, and all such modifications will be included in its scope. Similarly, there are solutions to these and other advantages and problems of the various embodiments as described above. However, the benefits, the advantages, solutions to problems, and any elements that can produce them or make them more explicit should not be interpreted as critical, required, or necessary one. The term “include”, “comprise” and any other variations thereof used herein are non-exclusive; accordingly, a process, method, article or device that includes a list of elements may include not only these elements, but also other elements that are not explicitly listed or are not part of said process, method, article or device. In addition, the term “coupling” and any other variations thereof as used herein may refer to physical, electrical, magnetic, optical, communication, functional, and/or any other connection.

Those skilled in the art will realize that many changes can be made to the details of the above embodiments without departing from the basic principles of the present disclosure. The scope of the present disclosure shall therefore be determined in accordance with the following claims.

1. An ultrasonic imaging system, comprising:
an ultrasonic probe having a plurality of array elements;
a transmitting and receiving control circuit configured to control the ultrasonic probe to transmit ultrasonic waves to a region of interest and receive ultrasonic echoes of the transmitted ultrasonic waves, to obtain channel echo data;
a processor configured to generate an ultrasonic image according to the channel echo data; and
a display configured to display the ultrasonic image;
wherein, the transmitting and receiving control circuit is configured to:
control the ultrasonic probe to transmit the ultrasonic waves at a plurality of steering angles to the region of interest, respectively; and
control the ultrasonic probe to receive the ultrasonic echoes of the ultrasonic waves transmitted at the plurality of steering angles, to obtain multiple groups of channel echo data, wherein each group of channel echo data is obtained by the ultrasonic echoes of the ultrasonic waves transmitted at one of the plurality of steering angles; and
wherein, the processor is configured to:
beam-form the multiple groups of channel echo data with a receiving-line grid comprising a plurality of grid points to obtain multiple groups of beam-formed data, wherein the multiple groups of channel echo data are beam-formed with a same receiving-line grid; and
generate the ultrasonic image based on the multiple groups of beam-formed data.

2. The ultrasonic imaging system according to claim 1, wherein, for one grid point of the receiving-line grid for one steering angle, the processor is configured to:
determines a receiving aperture based on a physical spatial position of the one grid point and the one steering angle; and
beam-form, based on the determined receiving aperture, the group of channel echo data obtained by the ultrasonic echoes of the ultrasonic waves transmitted at the one steering angle.

3. The ultrasonic imaging system according to claim 2, wherein the processor determining a receiving aperture based on a physical spatial position of the one grid point and the one steering angle comprises:
determining an intersection point of a straight line passing through the physical spatial position of the one grid point and forming an angle equal to the one steering angle with respect to a normal of the ultrasonic probe with the ultrasonic probe; and
determining the receiving aperture based on the intersection point.

4. The ultrasonic imaging system according to claim 1, the processor is further configured to perform a spatial compounding on the multiple groups of beam-formed data obtained by the beam-forming with the same receiving-line grid.

5. The ultrasonic imaging system according to claim 1, wherein the same receiving-line grid is a receiving-line grid corresponding to steering angles being 0.

6. The ultrasonic imaging system according to claim 1, wherein,
the processor generating the ultrasonic image based on the multiple groups of beam-formed data comprises: performing digital scan conversion on the beam-formed data to obtain ultrasonic image pixel data for display; and
the display is configured to display the ultrasonic image pixel data.

7. An ultrasonic imaging method, comprising:
transmitting, via an ultrasonic probe, ultrasonic waves at a plurality of steering angles to a region of interest, respectively;
receiving, via the ultrasonic probe, ultrasonic echoes of the ultrasonic waves transmitted at the plurality of steering angles, to obtain multiple groups of channel echo data, wherein each group of channel echo data is obtained by the ultrasonic echoes of the ultrasonic waves transmitted at one of the plurality of steering angles;
beam-forming the multiple groups of channel echo data with receiving-line grid comprising a plurality of grid points to obtain multiple groups of beam-formed data, wherein the multiple groups of channel echo data are beam-formed with a same receiving-line grid; and
generating an ultrasonic image based on the multiple groups of beam-formed data.

8. The ultrasonic imaging method according to claim 7, wherein beam-forming the multiple groups of channel echo data with receiving-line grid comprising a plurality of grid points comprises:
for one grid point of the receiving-line grid for one steering angle:
determining a receiving aperture based on a physical spatial position of the one grid point and the one steering angle; and
beam-forming, based on the determined receiving aperture, the group of channel echo data obtained by the ultrasonic echoes of the ultrasonic waves transmitted at the one steering angle.

9. The ultrasonic imaging method according to claim 8, wherein determining a receiving aperture based on a physical spatial position of the one grid point and the one steering angle comprises:
determining an intersection point of a straight line passing through the physical spatial position of the one grid point and forming an angle equal to the one steering angle with respect to a normal of the ultrasonic probe with the ultrasonic probe; and
determining the receiving aperture based on the intersection point.

10. The ultrasonic imaging method according to claim 7, wherein the same receiving-line grid is determined according to scan mode.

11. The ultrasonic imaging method according to claim 10, wherein:
the same receiving-line grid is a receiving-line grid under a Cartesian coordinate system when the scan mode is linear array scan mode; or
the same receiving-line grid is a receiving-line grid under a polar coordinate system when the scan mode is convex array scan mode or sector scan mode.

12. The ultrasonic imaging method according to claim 7, wherein the same receiving-line grid is a receiving-line grid corresponding to the steering angles being 0.

13. The ultrasonic imaging method according to claim 7, further comprising performing a spatial compounding on the multiple groups of beam-formed data obtained by the beam-forming with the same receiving-line grid.

14. The ultrasonic imaging method according to claim 13, wherein performing a spatial compounding on the multiple groups of beam-formed data obtained by the beam-forming with the same receiving-line grid comprises:
for one grid point of the same receiving-line grid, performing spatial compounding on the multiple groups of beam-formed data by averaging, weighted averaging, taking a maximum value or taking a median value.

15. The ultrasonic imaging method according to claim 8, wherein generating an ultrasonic image based on the multiple groups of beam-formed data comprises:
performing digital scan conversion on the beam-formed data to obtain ultrasonic image pixel data for display.

16. A non-transitory computer-readable storage medium comprising a program, which when executed by a processor, causes the processor to:
beam-form multiple groups of channel echo data with receiving-line grid comprising a plurality of grid points to obtain multiple groups of beam-formed data, the multiple groups of channel echo data being obtained by transmitting ultrasonic waves at a plurality of steering angles and receiving ultrasonic echoes of the ultrasonic waves transmitted at the plurality of steering angles, and each group of channel echo data being obtained by the ultrasonic echoes of the ultrasonic waves transmitted at one of the plurality of steering angles, wherein the multiple groups of channel echo data are beam-formed with a same receiving-line grid; and
generate an ultrasonic image based on the multiple groups of beam-formed data.
